(19) United States
(12) Patent Application Publication
(10) Pub. No.: US 2023/0218923 A1
(43) Pub. Date: Jul. 13, 2023

(54) PHOTODYNAMIC ANTIMICROBIAL THERAPY DEVICE

(71) Applicant: UNIVERSITY OF MIAMI, Miami, FL (US)

(72) Inventors: Jean-Marie A. Parel, Miami, FL (US); Cornelis J. Rowaan, Miami, FL (US); Alex Gonzalez, Miami, FL (US); Juan Silgado, Miami, FL (US); Alejandro Arboleda, Miami, FL (US); Guillermo Amescua, Miami, FL (US); Darlene Miller, Miami, FL (US); Heather Durkee, Miami, FL (US); Mariela Aguilar, Miami, FL (US)

(21) Appl. No.: 17/915,971

(22) PCT Filed: Mar. 29, 2021
(86) PCT No.: PCT/US2021/024683

§ 371 Date: Sep. 29, 2022

Related U.S. Application Data
(60) Provisional application No. 63/002,150, filed on Mar. 30, 2020.
(60) Provisional application No. 63/004,824, filed on Apr. 3, 2020.

(51) Int. Cl. A61N 5/06

(52) U.S. Cl. CPC A61N 5/062; A61N 5/0624; A61N 2005/0663; A61N 2005/0626; A61N 2005/0652; A61N 2005/0633

(57) ABSTRACT

Embodiments of an improved photodynamic therapy device are provided. An example of the device includes an irradiation head having a first end and a surface at a second end, the surface having a radius of curvature. The device also includes a plurality of light sources disposed on the curved surface. The plurality of light sources are configured to emit light having at least one wavelength corresponding approximately to an excitation peak of at least one photosensitizer. The light emitted by the plurality of light sources is focused to a focal point based on the radius of curvature of the surface and a target surface (e.g., a corneal surface of an eye) may be positioned at the focal point for treatment.

### CROSS-REFERENCE TO RELATED APPLICATIONS

This application claims priority under 35 U. S. C. § 119 (e) to U.S. Provisional Patent App. No. 63/002,150, filed on Mar. 30, 2020, which is hereby incorporated herein by reference as if set forth in full. This application also claims priority under 35 U. S. C. § 119 (e) to U.S. Provisional Patent App. No. 63/004,824, filed on Apr. 3, 2020, which is hereby incorporated herein by reference as if set forth in full.

### BACKGROUND

### Field of the Invention

The embodiments described herein are generally directed to ocular therapy, and, more particularly, to a portable light source for controlled delivery of wavelengths of light for use in various ocular therapy applications. Embodiments herein are also generally directed to photosensitizer solutions and methods for treating corneal and scleral infections, such as infectious keratitis.

### Description of the Related Art

Photodynamic therapy (PDT) involves the use of light to excite a photosensitizing chemical substance, which produces reactive molecular oxygen radicals to elicit cell death (phototoxicity).

PDT can be used to treat a wide range of medical conditions, including wet age-related macular degeneration, psoriasis, and atherosclerosis. This technology has also shown some efficacy in anti-viral treatments, including herpes. PDT also treats malignant cancers including head and neck, lung, bladder and skin. The technology has also been tested for treatment of prostate cancer, both in a dog model and in human prostate cancer patients. PDT can also be used for the treatment of various ocular conditions, such as corneal and scleral infections such as keratitis, skin infections and early melanomas, and treatment of ocular squamous neoplasia (OSSN) (ophthalmologists). The photo-chemical reaction of applying Riboflavin drops to the cornea stroma after removing the corneal epithelium and then activating the riboflavin with UV light (e.g., lamp emitting light having approximately 365-nm wavelength) is currently used as the standard of care for patients with progressive keratoconus and other forms of corneal ectasia. This is another example of a clinical application of PDT and is mostly called by the ophthalmic community by the term corneal crosslinking (CXL).

PDT may be both minimally invasive and minimally toxic. Other light-based and laser therapies such as laser wound healing and rejuvenation, or intense pulsed light hair removal do not require a photosensitizer. Photosensitizers have been employed to sterilize blood plasma and water in order to remove blood-borne viruses and microbes. PDT provides advantages that lessen the need for delicate surgery and lengthy recuperation and minimal formation of scar tissue and disfigurement. A side effect is the associated photosensitization of skin tissue.

Thus, there remains a need for improved PDT systems that are capable of delivering light to effectively treat a target area, while minimizing damage to healthy tissue.

### SUMMARY

Accordingly, embodiments of an improved photodynamic therapy device are disclosed. The following summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned herein.

In an embodiment, the device for photodynamic therapy treatment of a target biological tissue having an infection, the device comprises an irradiation head comprising a first end and a surface at a second end, the surface having a radius of curvature. The device also includes a plurality of light sources disposed on the curved surface. The plurality of light sources are configured to emit light having at least one wavelength corresponding approximately to an excitation peak of at least one photosensitizer. The light emitted by the plurality of light sources is focused to a focal point based on the radius of curvature of the surface.

In an embodiment, a photodynamic therapy system is disclosed. The photodynamic therapy system comprises a device for photodynamic therapy treatment of a target biological tissue and a controller communicatively coupled to the plurality of light sources and configured to control the light emitted by the plurality of light sources. The device comprises an irradiation head comprising a first end and a surface at a second end, the surface having a radius of curvature. The device also includes a plurality of light sources disposed on the curved surface. The plurality of light sources are configured to emit light having at least one wavelength corresponding approximately to an excitation peak of at least one photosensitizer. The light emitted by the plurality of light sources is focused to a focal point based on the radius of curvature of the surface.

In an embodiment, a method of treating a condition of a target biological tissue is disclosed. The method comprises deploying a photodynamic therapy system, the system comprising a device for photodynamic therapy treatment of a target biological tissue and a controller communicatively coupled to the plurality of light sources and configured to control the light emitted by the plurality of light sources. The device comprises an irradiation head comprising a first end and a surface at a second end, the surface having a radius of curvature. The device also includes a plurality of light sources disposed on the curved surface. The plurality of light sources are configured to emit light having at least one wavelength corresponding approximately to an excitation peak of at least one photosensitizer. The light emitted by the plurality of light sources is focused to a focal point based on the radius of curvature of the surface. The method also comprises positioning the device relative to the target biological tissue such that the target biological tissue is positioned at the focal point of the radius of curvature; applying the at least one photosensitizer to the target biological tissue, the at least one photosensitizer having the excitation peak; and controlling the plurality of light sources, using the controller, to emit light having the at least one wavelength toward the photosensitizer, thereby exciting the photosensitizer and producing oxygen radicals.

In an embodiment, a method of treating a condition of an eye is disclosed. The method comprises applying at least one photosensitizer to the cornea of an eye, the photosensitizer having an excitation peak; positioning a corneal surface of the eye at a focal point of a cupula; and applying at least one wavelength of light to the cornea of the eye for a predetermined period of time using a plurality of light sources emitting light having the at least one wavelength that corresponds approximately to the excitation peak of the at least one photosensitizer, the plurality of light sources housed in the cupula structure, the cupula structure having a radius of curvature that focuses the light emitted from each of the plurality of light sources at the focal point and on to the corneal surface of the eye positioned at the focal point of the cupula.

In yet another embodiment, a device for photodynamic therapy is disclosed. The device comprises a cupula structured member having a radius of curvature; and a plurality of light sources positioned within a concave surface of the cupula structured member. Each of the plurality of light sources configured to emit light of a wavelength that is common amongst the plurality of light sources, the wavelength corresponding to an excitation peak of a photosensitizer. The radius of curvature of the cupula focuses light emitted by the plurality of light sources to a focal point

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a graph depicting example photosensitivity treatment spectra;

FIG. 2A illustrates an example photodynamic therapy system in accordance with embodiments herein;

FIG. 2B illustrates the example photodynamic therapy system of FIG. 2A in operation;

FIG. 3A illustrates an example photodynamic therapy device in accordance with embodiments disclosed herein;

FIG. 3B illustrates an example irradiation head for the photodynamic therapy device of FIG. 3A;

FIGS. 4A-4C illustrate various views of another example irradiation head, with the outer housing removed;

FIGS. 5A-5C illustrate various views of another example irradiation head, with the outer housing removed;

FIGS. 6A-6D illustrate an example graphical user interface for controlling the photodynamic therapy system of FIG. 2A; and

FIG. 7 is a schematic block diagram illustrating an example wired or wireless computer system according to embodiments of the present disclosure.

Various embodiments disclosed herein are described in detail with reference to the aforementioned figures. The drawings are provided for purposes of illustration only and merely depict example embodiments. These drawings are provided to facilitate the reader's understanding and shall not be considered limiting of the breadth, scope, or applicability of the embodiments. It should be noted that for clarity and ease of illustration these drawings are not necessarily made to scale.

### DETAILED DESCRIPTION

The detailed description set forth below, in connection with the accompanying drawings, is intended as a description of various embodiments and is not intended to represent the only embodiments in which the disclosure may be practiced. The detailed description includes specific details for the purpose of providing a thorough understanding of the embodiments. However, it will be apparent that those skilled in the art will be able to understand the disclosure without these specific details. In some instances, well-known structures and components are shown in simplified form for brevity of description. Some of the surfaces have been left out or exaggerated for clarity and ease of explanation.

Reference throughout this disclosure to “embodiment” or “example” or “iteration” means that a particular feature, structure, or characteristic described in connection with the embodiment is included in at least one embodiment. Thus, appearances of the terms “embodiment” or “example” or “interaction” in various places throughout this specification are not necessarily all referring to the same embodiment. Furthermore, the particular features, structures, or characteristics may be combined in any suitable manner in one or more embodiments.

PDT involves three components: a photosensitizer, a light source, and tissue oxygen. The wavelength of the light source needs to be appropriate for exciting the photosensitizer to produce radicals and/or reactive oxygen species. These are free radicals generated through electron abstraction or transfer from a substrate molecule and highly reactive state of oxygen known as singlet oxygen. PDT is a multi-stage process. First a photosensitizer with minimal dark toxicity, in some embodiments, is administered to a target area (e.g., diseased tissue), either systemically or topically. When a sufficient amount of photosensitizer is applied to the target area, light is applied to the photosensitizer for a specified period, which activates the photosensitizer. Activating the photosensitizer produces radicals and/or reactive oxygen species, which elicit cell death of the diseased tissue. The light exposure supplies sufficient energy to stimulate the photosensitizer, but the dose period and energy levels are controlled so to not damage neighboring healthy tissue.

Embodiments presented herein provide for systems and methods for PDT that utilize a unique portable source of light emitting wavelengths that correspond to (or close to) excitation peaks of photosensitizers using a series of light sources (such as, but not limited, to miniature light-emitting diodes or LEDs, multispectral LEDs, and the like). For example, in some implementations embodiments herein emit light having a wavelength within +/−25 nm of the excitation peak of the photosensitizer. In another example, the light may be +/−15 nm. In some embodiments, the light sources may be tunable LEDs configured to produce multispectral output. Tunable LEDs maybe controlled (e.g., by a controller) to selectably emit a desired spectral wavelength. Embodiments herein provide a plurality of light sources that emit light at wavelengths to excite one or more photosensitizers. In some embodiments, the light sources may be lasers, laser diodes, and/or other sources of white light with a bandpass filter that filters the light to correspond to the excitation peak of the photosensitizer. The light sources are arranged to focus the emitted light at a single focal point or area. In various embodiments, the light sources are housed in a cupula having a radius of curvature that focuses the light emitted from each individual light source on to the focal point of the curvature of the cupula. That is, for example, the cupula supports the plurality of light sources and is shaped to direct each light source toward the eye. The radius of curvature defines the distance from the cupula to the eye. The radius of curvature of the cupula may be based on the divergence of the beam from the light sources and the angle at which the light is emitted relative to a central axis. Such physical properties of the emitted light may be used to cover a defined circular area at a distance to the eye. In an example implementation, the radius of curvature was designed to be 75 mm, but the radius can be anything one selects as it may be a function of the divergence angle of the light and manufacturing precision of the light sources (e.g., LEDs).

Embodiments described herein may be applied to photodynamic therapy (PDT) of corneal and scleral infections (ophthalmologist, optometrists) such as keratitis, PDT of skin infections and early melanomas, and treatment of ocular squamous neoplasia (OSSN) (ophthalmologists). In some implementations, a target tissue (e.g., a corneal surface in some examples) may be positioned at the focal point of the cupula. As used herein, tissue refers to biological tissue. In another example, embodiments herein may be used to crosslink corneal collagen fibers and make the cornea stronger.

Embodiments herein provide numerous, non-limiting advantages. For example, embodiments disclosed herein reduce concerns about antimicrobial resistance, because the microbes don't have capacity to create resistance to this type of therapy. Additionally, embodiments herein also improve patient compliance, because the treatment can be delivered in an office/clinical setting.

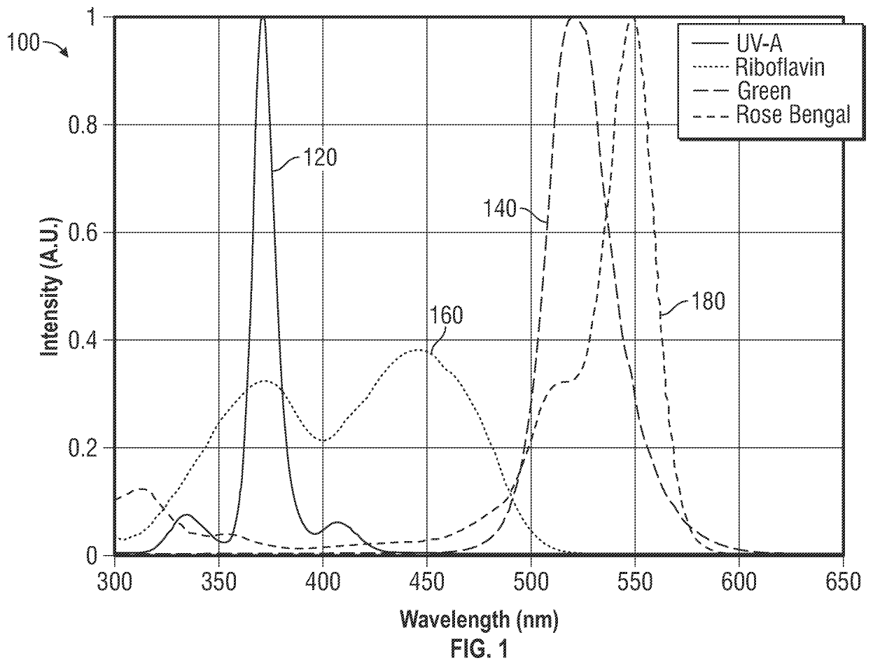

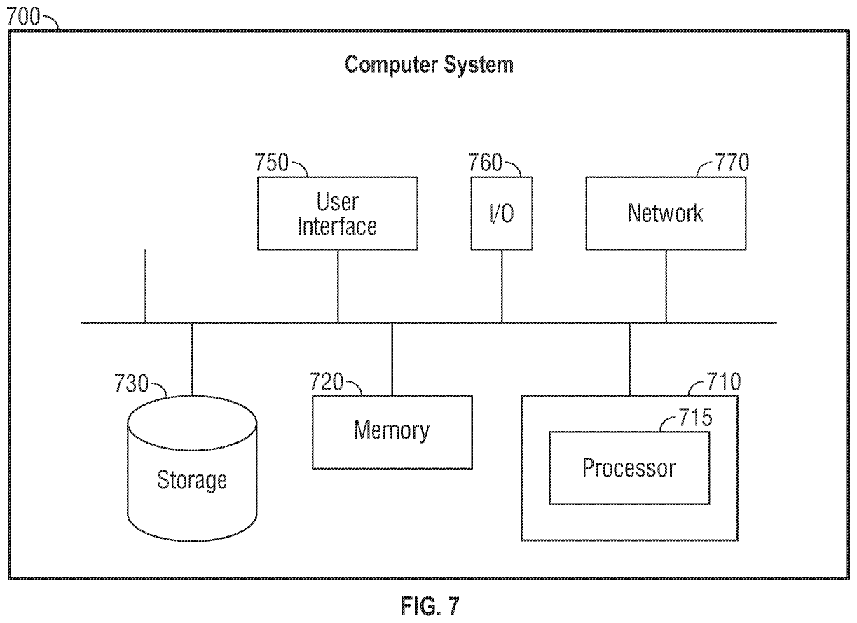

Example photosensitizers include dyes and stains, such as but not limited to, Rose Bengal and Erythrosin B, each of which have an excitation peak of approximately 525 nm (see FIG. 1). Other example stains and/or dyes having different and/or the similar excitation peaks are within the scope of this disclosure. In some examples, the cupula may house additional or alternative light sources (such as, but not limited to, miniature LEDs and the like) emitting light at a wavelength to excite other photosensitizers, such as but not limited to, riboflavin having an excitation peak at approximately 375 nm (see FIG. 1), dihematoporphyrinether at having an excitation peak at less than or approximately equal to 400 nm (e.g., Soret band) and/or an excitation peak of approximately 670 nm, and photosensitizers that can be excited in the Soret band such as verteporfin used in ophthalmology (e.g., an excitation peak of approximately 395 nm). The source spectra of the light sources can be adjusted to any available photosensitizer and embodiments herein can be programmed to activate light sources of a corresponding excitation peak so to excite respective photosensitizers via software running on a controller (e.g., a computer executing instructions stored on a memory, such as that described in connection to FIG. 7). The light sources may be powered by electronics under control of the controller.

FIG. 1 is a graph 100 depicting an example photosensitivity treatment spectra. FIG. 1 shows intensity of light in arbitrary units (A.U.) for multiple spectra as a function of wavelength in nanometers (nm). Lines 120 and 140 represent light source emission spectra. Line 120 represents a spectra of light emitted by a light source emitting light having a UV-A spectra and line 140 represents the spectra of light emitted by a light source having a green spectra. Lines 160 and 180 represent absorption spectra for two example photosensitizers. Line 160 represents the absorption spectra of riboflavin and line 180 represents the absorption spectra of Rose Bengal. Thus, based on the absorption spectra of a selected photosensitizer, light sources may be utilized in the embodiments disclosed herein configured to emit light having a wavelength spectra corresponding to the excitation peaks of the photosensitizer. That is, the light sources may be controlled to emit light having a peak wavelength that is approximate to or close to the excitation peak of the photosensitizer, as shown in FIG. 1.

In some embodiments, photosensitizers may be formulated as a film to be applied to target tissue. In another example, photosensitizers may be provided as a gel (e.g., similar to how riboflavin is currently provided by Avedro, Inc.).

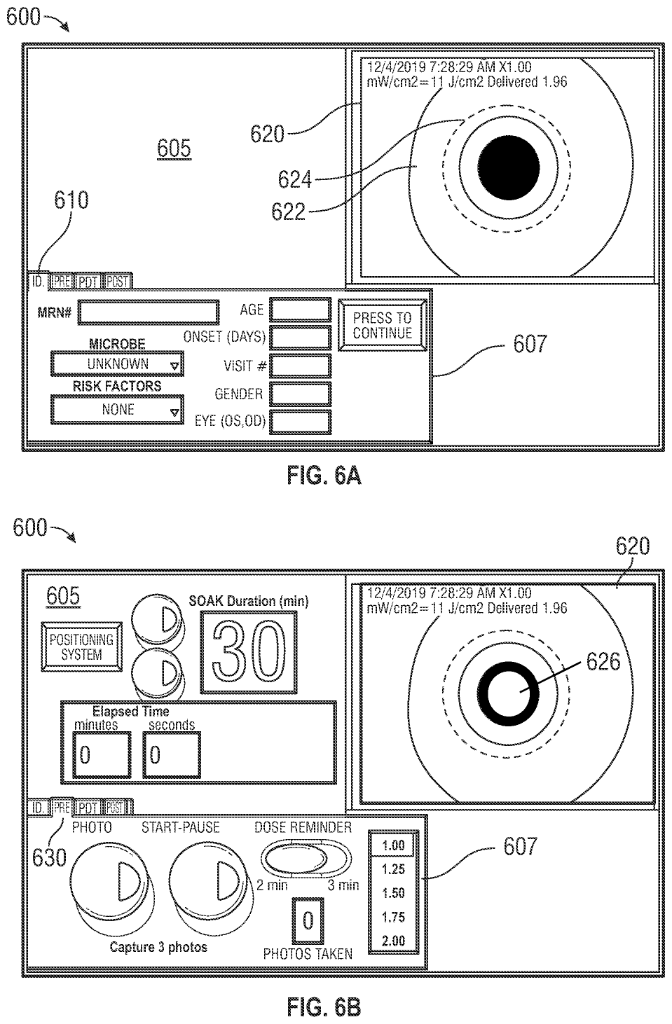

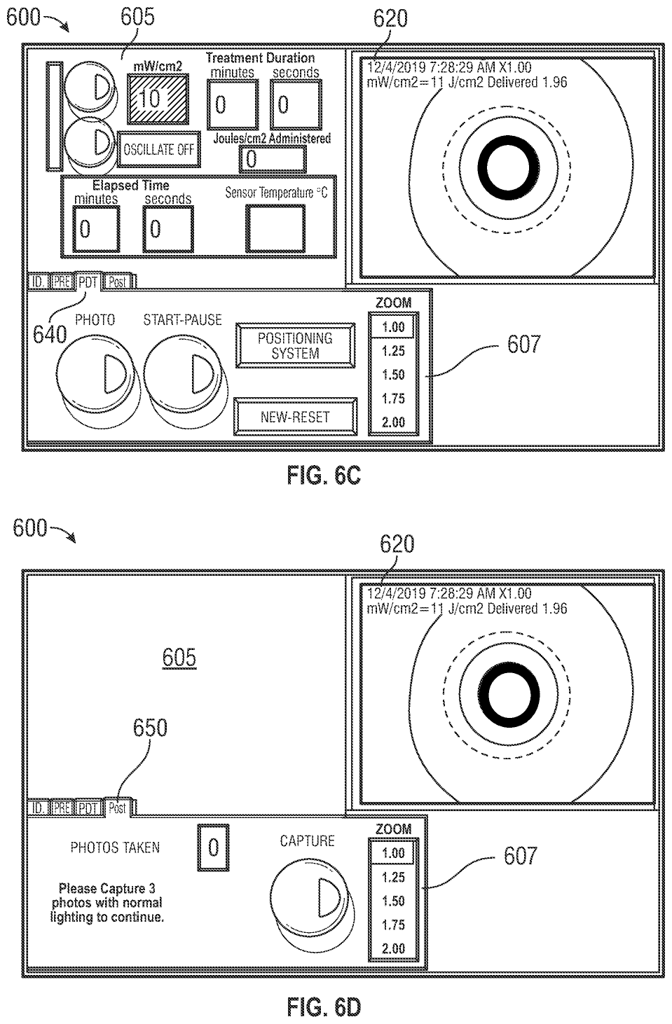

An illustrative Rose Bengal formulation comprises 4 sterile strips of Rose Bengal (HUB Pharmaceuticals) immersed in a vial containing 5 cc of saline (0.9% NaCl) or BSS shaken vigorously for 1 minute generating a 0.1% RB solution and transferred to a syringe by aspiration and deposited on both sides of a clinically available sponge (e.g., 8 mm corneal sponge, BeaverVisitec International, Waltham, Mass., USA). The sponge may be placed on the center of the corneal or scleral defect of an eye for treatment. The sponge soaked with the photosensitizer may be left on the cornea for 30 minutes while 2-3 drops of Rose Bengal solution are placed on the sponge every 2 or 3 minutes when the console (e.g., the controller) issues audible or visual notifications (e.g., beeps/other audio notification and/or lights from the controller). The timing of the notifications may be adjustable via a user interface on the controller (see FIGS. 6A-6D). Should a 0.2% Rose Bengal solution be needed, only 2.5 cc of saline or BSS are placed in the vial.

Another example formulation is to add the appropriate weight of Rose Bengal in a dark brown glass vial sealed with a rubber septum which is autoclaved for sterility. Then 5 or 2.5 cc of the above described fluids may be added to generate a 0.1 or 0.2 Rose Bengal solution. The same or similar procedure can be used for riboflavin and any other photosensitizers that are available in powder form. In each case, a 0.2 μm filter may be used to aspirate the solution via a sterile syringe to prevent extraneous matter from contaminating the solution.

As several light sources can be used, in an example implementation, some embodiments herein comprise a first plurality of light sources capable of emitting light having a first wavelength to excite a first photosensitizer and a second plurality of light sources, different from the first light sources, capable of emitting light having a second wavelength, different from the first wavelength, to excite a second photosensitizer. In such a configuration, where the target tissue, for example, the corneal of surface of an eye, is located at the focal point of the cupula, the optics of the eye will only be able to image a single light source on the fovea and thereby limiting the focal intensity well below the accepted irradiance safety level for a 24 hrs exposure. As an example, 13 to 15 light sources can be used as the first plurality of light sources emitting light at a wavelength to excite Rose Bengal as the first photosensitizer and a separate 13 to 15 light sources as the second plurality of light sources for riboflavin as the second photosensitizer.

The radius of curvature of the cupula can be varied. Varying the radius of curvature may refer to a cupula having a plurality of surface regions on which light sources are mounted, each region having a different radius of curvature. Thereby providing multiple focal points for the respective light sources. Varying the radius of curvature allows the light sources to be positioned at multiple distances from the target (e.g., the eye) and vary the energy delivered. Alternatively, varying the radius allows the light sources for each region to emit light incident at surfaces of varying distance away from the cupula. As an illustrative example, if PDT were required to cover rumors/growth on the nose, the cupola could be formed of varying curvature so a first region having a first group of light sources could cover the tip of the nose, a second region having a second group and third region having a third group of light sources for each side of the nose, and a fourth region having a fourth group of light sources for the base of the nose. Additionally, more than groups of light sources could be used to focus at different distances from the tip of the nose to its base. Similarly, as the eye is curved, a different regions with different groupings of light sources may be used to focus light onto different regions of the eye to achieve a distributed irradiation without needing to readjust the cupula relative to the eye. As used herein, each region may comprise a row of light sources, a column of light sources, and/or any collection of light sources as long as each region comprises fewer than all of the light sources of housed in the cupula. In an illustrative example, a focal distance of 75 mm was used for the radius of curvature, which gave the of the device operator ample free space to irrigate the corneal surface to prevent desiccation of the tissues during treatment while killing microbes. However, any desired focal distance may be used as desired by the specific application. For example, a radius of curvature of less than 75 mm may be used for treatment of skin infections on hands, legs, torso, back, face, etc. of a patient.

In some embodiments, a triangulation system may be included to assist with placement of the light source relative to the target tissue (e.g., the corneal surface of the eye or some other target surface), such that the target tissue is properly aligned the focal point. An example triangulation system may include a plurality of coherent light sources (e.g., laser diodes) located equidistant from a central axis of the light sources. For example, equidistant from a central axis of the cupula, such as at the rim of the cupula or at another radius from the central axis. The plurality of coherent light sources may include three or more coherent light sources, such as miniature low power visible spectrum laser diodes. The plurality of coherent light sources may be aligned such that the emitted beams converge at a single focused spot at the focal point of the cupula. While not necessary, it may be preferred that wavelength of the beams be within the visible spectrum to facilitate visualization by the operator. For example, while positioning the cupula relative to the target tissue (e.g., an eye or other tissue for treatment), proper placement of the cupula may be determined when the target surface is positioned relative to the cupula such that the beams converge at a single spot on the target. However, other wavelengths may be used in conjunction with corresponding detection techniques to detect proper alignment and convergence of the light emitted from the coherent light sources. In some embodiments, the laser power may be restricted to be 1 mW or less. As the coherent light sources have their own distinct optical elements, these elements may be configured to focus the beams on the cornea of the eye. Thus, when the cupula is properly positioned, none of the beams are focused on the fovea of the eye together or singly thereby avoiding tissue damage even during an exposure of 24 hrs.

In various embodiments, the light sources and coherent light sources described herein may be lower power. By utilizing low power light sources, a small and lightweight console may be powered by 90 to 260 VAC wall supply or by an internal rechargeable battery may be used. The console may contain the electronic circuitry and a subminiature PC (or controller) to allow the operator to enter subject data type of infection (if known) and program treatment parameters, along with electronic circuitry and components to control light sources, triangulation systems, etc. In some embodiments, the controller, such as the computer system represented in FIG. 7, controls the operation of the system. From the console, the light source output energy may be adjustable from approximately 3 to approximately 18 mW/cm² for Rose Bengal and approximately 3 to approximately 15 mW/cm² for Riboflavin, other energies may be utilized based on the respective photosensitizer. These boundaries may be constrained in time to limit the treatment time when a threshold energy is reached. For example, a threshold of 5.4 mW/cm2 under the current standards as set by the medical community and/or FDA. While certain examples are provided above, it will be appreciated that the output energy parameters are adjustable should the standard of care be revised and different photosensitizers be utilized. That is, the source output energy and the treatment time, upon exceeding the threshold, maybe dependent on the specific photosensitizer used as outlined above.

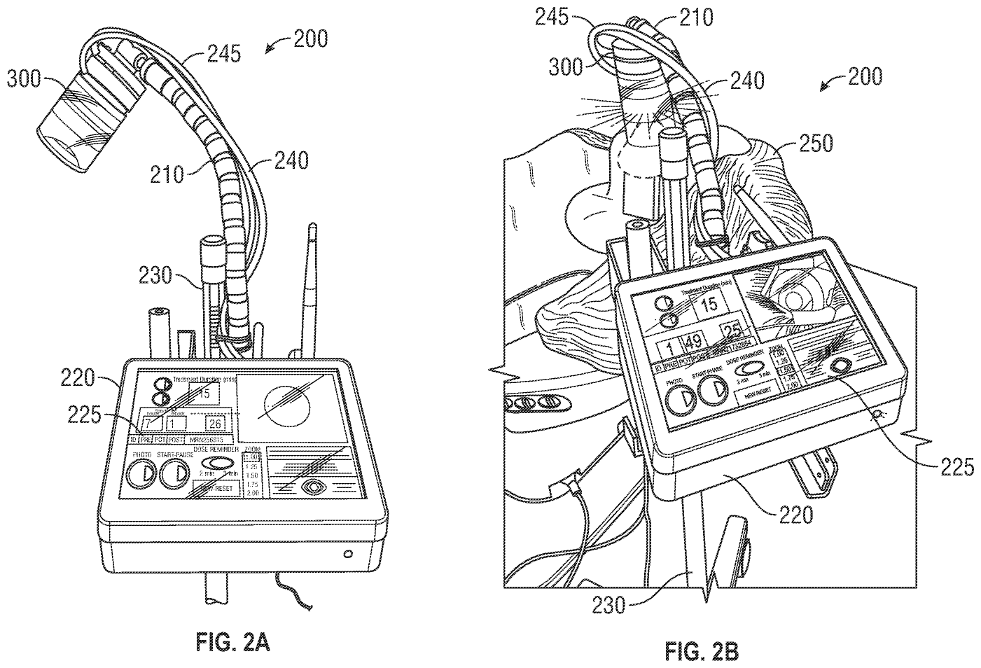

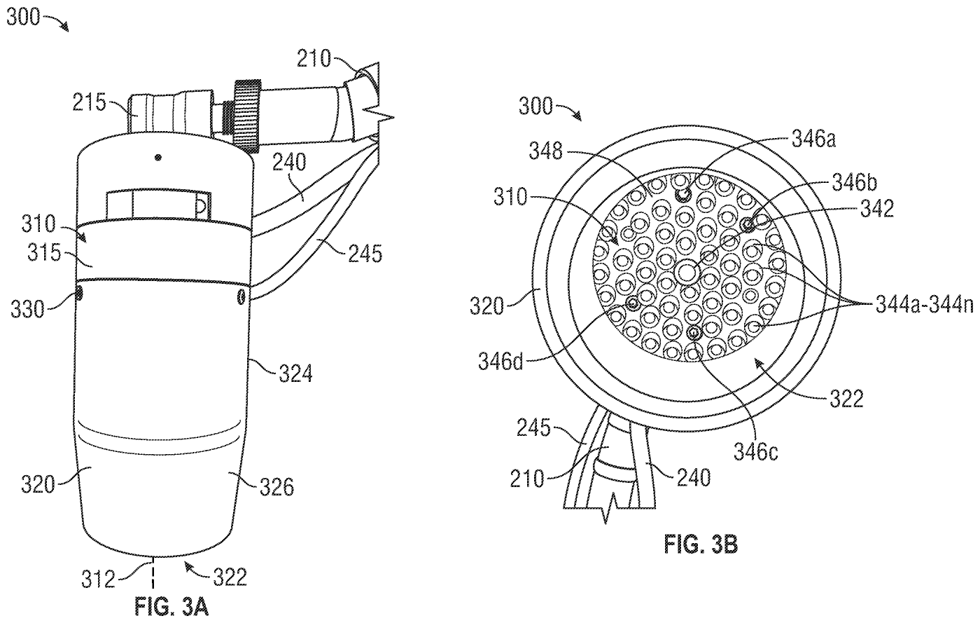

FIGS. 2A and 2B illustrate an example photodynamic therapy (PDT) system 200 (referred to herein as system 200) in accordance with embodiments herein. The system 200 may include a controller 220 comprising a user interface 225, a support member 210, and a PDT device 300. FIG. 2A illustrates the system 200 generally and FIG. 2B illustrates the PDT system 200 in operation such that the cupula (see FIGS. 3A and 3B) of the PDT device 300 is positioned with respect to an target tissue, such as an eye of patient 250 in this illustrative example.

The controller 220 may be implemented as a computer system, such as the computer system 700 described in connection to FIG. 7 housed in a console. The controller 220 may be coupled to a user interface 225 and a display (such as a LCD display, OLED display, or the like). In the illustrative example, the user interface 225 is a touchscreen display on which a graphical user interface is generated (see FIGS. 6A-6D) and configured to receive user inputs for controlling the system 200. However, the user interface may be from other means, such as a mouse and keyboard, gesture tracking of operator hand movements, remote input devices such as tablets, mobile phones, etc.). The controller 220 is communicatively coupled to the PDT device 300 via a wired or wireless connection. In the example shown in FIGS. 2A and 2B, the controller 220 is connected to the PDT device 300 via wire 240 for transmitting control signals from the controller 220 to the PDT device 300 and wire 245 supplies power to the PDT device 300 from a power source (e.g., a battery or wall outlet). Wires 240 and 245 may be combined into a single wire and other wires maybe included, such as wires for controlling a triangulation system and/or an image capture device included in the PDT device 300). In some examples, one of wires 245, 240 may include a multi female receptacles socket coupled to the controller 220. Each PDT device 300 may have a multi prong male connector that mates with the multi female receptacle socket for establishing electrical connections with the controller 220. The multi prong male connector may include a pin that indicates to the controller 220 what wavelength(s) the irradiation head is designed to produce. The controller 220 may determine the wavelength(s), identify the proper voltage and software for the particular irradiation head, and generate and apply the voltage and display (e.g., via user interface 225) the software. Different irradiation head may have different number, types, etc. of light sources, and the voltage and current required to drive each irradiation head may vary. Thus, the controller 220 is able to recognize which irradiation head is connected and drive the irradiation head properly.

In the illustrative example of FIGS. 2A and 2B, the system 200 is attached to an IV pole 230, which may facilitate ease of positioning the system 200. The system 200 may be lightweight, portable and designed to fit any IV pole, for example, in hospital and clinics for portability. As another example, system 200 could also be mounted on a table top instead of an IV pole for use in an operating room next to the operation microscope. Furthermore, the system 200 be placed on a slit lamp table next to the slit-lamp biomicroscope.

The support member 210 may be removably attached to the PDT device 300 and arranged to hold the PDT device 300 in a position. For example, the support member 210 may hold the PDT device 300 in a desired position above the patient 250. The support member 210 may be a flexible arm that permits positioning of the PDT device relative to the eye of the user. The PDT device 300 may be removably attached via mounting member 215, such as but not limited to, a threaded fastener (e.g., a screw), clip, spring loaded attachment, magnetic coupling, or the like. In an example implementation, the flexible arm may be an adjustable goose neck supporting the PDT device 300 and may be connected to the controller. In another embodiment, the flexible arm may be attached to one of a slit-lamp head rest bar, or plugged into the pivoting axis of a slit-lamp arm. The support 210 may also be an adjustable stereotactic clamp.

In another example, the support member 210 may be a motorized robotic arm. The motorized robotic arm may be controlled by the controller 220 via automated computer control (e.g., based on video tracking as described below) or remote control by an operator via inputs in the user interface 225. Control of the robotic arm may be able to continuously maintain a desired relative position such that the target tissue (e.g., the patient's infection) remains within the optimal energy zone of the device (see FIGS. 6A-6D) throughout the treatment. The continuous tracking and robotic movement may be able to compensate for patient movements for example by translating changes in relative position and moving the PDT device 300 based on the changes.

FIGS. 3A and 3B illustrate an example PDT device 300 in accordance with embodiments disclosed herein. The PDT device 300 comprises an irradiator head 310 that is removably coupled to the mounting member 215 at a first end and is coupled to a shield 320 at a second end. The irradiation head 310 houses a plurality of light sources 344a-344n (collectively referred to herein as light sources 344) that emit light toward an aperture 322 of shield 320. Target area of target tissue may be positioned within the aperture 322, such that light emitted from the light sources 344 is incident on the target area of the target tissue. The target area may comprise a photosensitizer that is excited based the wavelength of light emitted by the light source 344

The irradiation head 310 comprises an outer housing 315 and surface 348 at the second end of the irradiation head 310. The surface 348 may be removably attached to an outer housing 315 via fasteners 346a-346d (e.g., thread screws, rivets, etc.). The surface 348 has a plurality of openings in which a plurality of light sources 344a-344n can be mounted. The surface 348 be a curved surface having either a single or varied radius of curvature. The shield 320 extends from the second end of the irradiation head 310 in a direction opposite the mounting member 215 to an aperture 322. In various embodiments, the surface 348 is a concave surface curving in a direction away from the aperture 322.

The light sources 344 may be arranged any desired arrangement. For example, in a radial array of having a number of light sources arranged in a circular configuration at increasing radii. As another example (as illustrated in FIG. 3B), the surface 348 may be divided into three (or more) regions and the light sources 344 arranged such that the light source 344 are symmetrically arranged in each region. In another example, subsets of light sources 344 may be mounted at regions of surface 348 having different radii of curvature, such that the cupula has multiple focal points. For example, where there are three symmetrical regions of the surface 348, within each region there may be smaller regions each having a different radius of curvature and one or more light sources may be mounted in each smaller region. Thus, light sources mounted to each smaller region from each of the three regions may be focused at different focal points of the cupula. Thus, the cupula may have a varying radii (as described above), which allows the device 300 to be positioned at multiple distances from an eye and to vary the energy delivered.

For example, since the eyeball is curved and not flat, an illustrative example of the device 300 may comprise a central row of light sources (e.g., a first region having a first grouping of light sources) housed in the cupula and focused, based on the radius of curvature of the first region, on the center of the cornea. The device 300 may also include a last row (e.g., a second region having a second grouping of light sources) housed in the cupula and focused, based on the radius of curvature of the second region, at the periphery of the eye (e.g., past the cornea-limbus). Additionally, the device 300 may include one or more intermediate rows (e.g., one or more regions having one or more groupings of light sources) in between the central and last rows. Each intermediate row of light sources may be focused, based on the radius of curvature of each respective region, at locations between the center of the eye to approximately 3 mm below the limbus in which case the energy per mm² would be distributed approximately equally on the ocular surface.

In this illustrative example, the irradiation head 310 is cylindrical in shape and the shield 320 is at least partially cylindrical in shape (and may be entirely cylindrical in shape), both aligned along central axis 312. The irradiation head 310 and shield 320 may have an equal or substantially equal outer radius and the shield 320 may have an inner radius. However, the present disclosure is not so limited to only cylindrical shapes, the functions of the PDT device 300 described herein are achieved, any shape of irradiation head 310 and shield 320 may be utilized.

Accordingly, the shield 320 and the surface 348 may form a cupula housing light sources 344. Target area of target tissue may be positioned at a focal point of the cupula, within the aperture 322, such that light emitted from the light sources 344 is incident on the target area. In some embodiments, the surface 348 may be formed to reflect and direct light emitted from the light sources 344 toward the aperture 322 (more specifically to the focal point of the cupula). In another embodiment, which can be implemented separately and/or with the reflective nature of the surface 348, each light source 344 may comprise an optical system (e.g., lens and optical elements) arranged to focus the emitted light into a coherent beam directed toward the focal point. As another example, the light sources 344 may emit light having a desired half-angle such that the majority of emitted light is directed toward the focal point.

The irradiation head 310 may also house an image capture device 342 for capturing images and/or video streams (e.g., multiple sequential images) of the target area before, after and during operation. The image capture device 342 may be positioned at the center of the surface 348 along the axis 312. The image capture device 342 may be, for example, a micro-camera (e.g., one or more lenses that focus received light to an image sensor, such as CCD, CMOS, and the like). The image capture device 342 may be communicatively coupled to the controller 220 and controlled thereby for capturing images of the target tissue. In some embodiments, the image capture device 342 may capture images, transmitted to the user interface, for use in aligning and maintaining proper alignment of the PDT device 300 relative to the target area.

In various example, the image capture device 342 may be used in conjunction with or as part of a video tracking system that passive enables the operator to visualize the surface being treated. The video system may be configured, for example, to track eye positions and palpebral fissures opening, for situations where a speculum is not used and the patient either has their closed eyes, is fixating incorrectly, or has excessive blinking. In another example, the image capture device 342 may be used in conjunction with or as part of a triangulation system that enables the operator to visualize proper alignment.

The irradiation head 310 may be coupled to the controller 210 via wired or wireless connection for receiving control signals to control the light sources 344 and supply power thereto. The irradiation head 310 may also house electronics, such as a PC board, and heat exchange systems (e.g., cool fans and heat syncs) for controlling the heat generated by the electronics and light sources housed therein. In some examples, the irradiation head 310 may be passively ventilated via openings permitting air flow to cool the cupula. In another example, alone or in combination with passive ventilation, the irradiation head 310 may be cooled via the fan under control by the controller to regulate the internal temperature of the irradiation head 310 to maintain optimal temperature and power output. In either example, the controller may be configured to alert the operator via audio and/or visual alerts, if the optimal head temperature is not maintained and the device is operating out of range.

The shield 320 maybe removably coupled to the irradiation head 310 via fasteners 330. As illustrated in FIG. 3A, the shield 320 may have a radius that is varied along the length of central axis 312. For example, the shield may include an upper portion 324 having a constant radius forming a cylindrical shape and a lower portion 326 having a varied radius forming a truncated conical shape. In another example, the shield may have a constant radius along the entire length. In yet another example, the radius of the shield may be varied along the entire length.

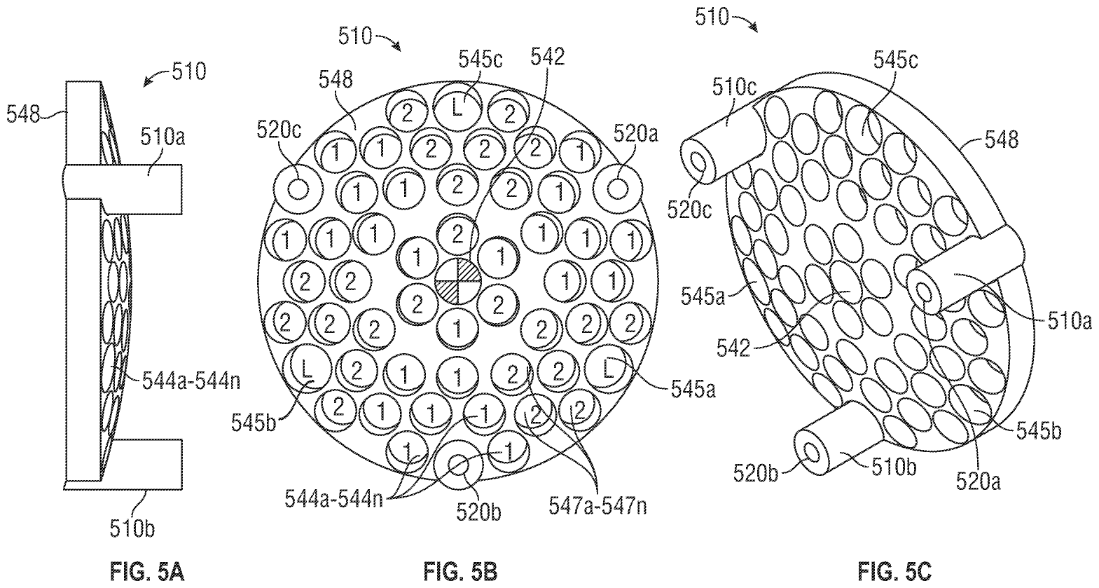

In the illustrative example of FIG. 3B, the device 300 is equipped with a selection of light sources that produce a light of a single peak wavelength. In another example, the device may comprise two, three, or more types of light sources each type producing different peak wavelengths (examples of which are shown in FIGS. 5A-5C) each type controlled independently, permitting different photosensitizers to be used in treatment.

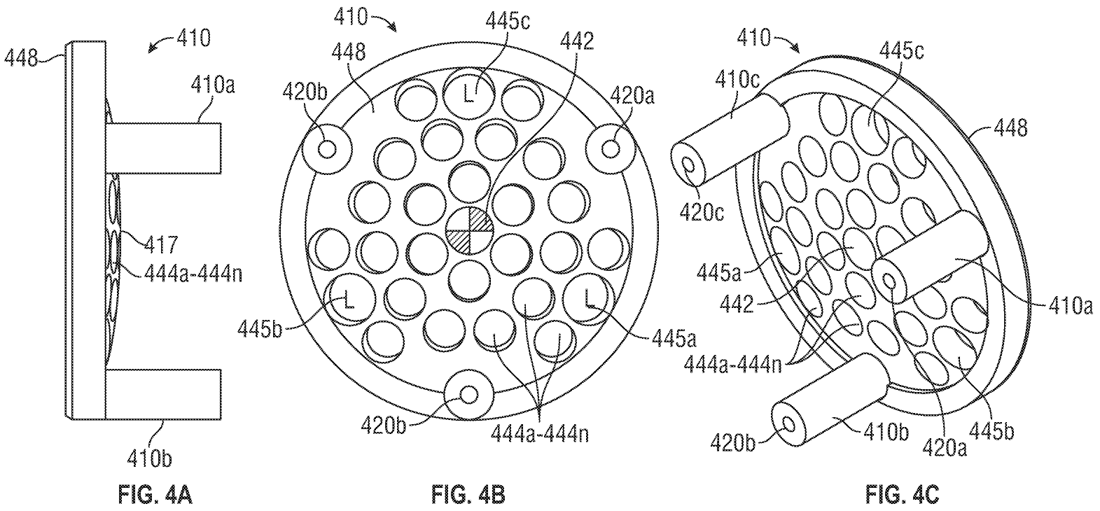

In some embodiments, the device 300 (and thus the cupula) may be positioned at the required distance from the target are (e.g., the eye) via a measuring device such as a ruler or caliper. In another example, the distance between the device 300 and the target area may be estimated using a triangulation system (sometimes referred to herein as a triangulation positioning system or laser triangulation positioning system), where beams converge to a single point when the device 300 is located at a proper position relative to the target area (examples of which are shown in FIGS. 4A-5C). In another example, the device may use a sonar distance gauge, to guide the operator when the correct tissue to cupula distance has been achieved.

In the embodiments described herein, light emitted from the light sources 344 are centrally focused along the central axis 312 at a focal distance of the cupula. When used as part of system 200, the support 210 may permit the irradiation head 310 to be positioned in XYZ axis. The operator may be guided by the image capture device 342 that is colinear with the cupula (e.g., positioned along the central axis 312) and outlines the optimal energy delivery zone (e.g., as illustrated in FIGS. 6A-6D). In some examples, the image capture device 342 may facilitate visual inspection of the triangulation system to confirm convergence for proper positioning. In another example, the operator may identify a treatment region and the device 200 may track the region of interest to maintain an ideal position.

In some embodiments, the irradiation head 310 may be fixed, emitting only light of the light sources housed therein. In another example, the irradiation head 310 may be swappable (e.g., removable), via releasable mounting member 215, so to switch between irradiation heads depending on the microorganism identified and the photosensitizer desired for treatment.

As described above, embodiments herein may comprise light sources that generate 525 nm light to be used with several photosensitizers, such as, but not limited to, Rose Bengal, Erytrosin B, and Methylene Blue. The light sources may also emit 400 nm light that can be used to excite all photosensitizers excitable in the Soret band, such as, but not limited to, hematoporphyrins derivatives including photofrin and Verteporfin. Furtherer still, the light sources may emitter 375 nm UV-A light for corneal crosslinking using riboflavin photosensitizer.

In an illustrative implementation shown in FIG. 3B, the irradiation head 310 may comprise 54 LEDs housed in a 100 mm focal length cupula. Each LED may be configured to emit green light (e.g., a peak wavelength at 525 nm) having a 45 degree half-angle. In this example, the irradiation head may produce light having an incident energy up to 6 mW/cm². While the above embodiment is described with reference to an example configurations, the scope of this disclosure is not limited to these specific examples. For example, the number of light sources housed in the cupula and the power emitted thereby may be dependent on the size of the individual light sources and the size of the cupula and the desired incident power at the target tissue.

FIGS. 4A-4C illustrate an example irradiation head 410 in accordance with embodiments disclosed herein, but with the outer housing removed. The irradiation head 410 is substantively the same as the irradiation head 310. Therefore, the description above with respect to FIGS. 3A and 3B apply equally to the embodiments shown in FIGS. 4A-4C, unless conflicting description is provided herein.

Similar to the irradiation head 310 of FIGS. 3A and 3B, the irradiation head 410 includes a surface 448. As shown in the example of FIGS. 4A-4C, the surface 448 has a concave shape, however other shapes are possible as set forth above. Irradiation head 410 also comprises a plurality of light sources 444a-444n mounted to the surface 448 in a desired array and an image capture device 442 centrally mounted to the surface 448. The light sources 444a-444n and image capture device 442 may be substantively the same as light source 344 and image capture device 342, respectively. FIGS. 4A-4C illustrate through holes 420a-420c and a cylindrical posts 410a-410c for receiving fasteners to mount the irradiation head 410 to the outer housing (e.g., outer housing 315 of FIG. 3A).

Additionally, the irradiation head 410 comprises a plurality of coherent light sources, for example, a plurality of laser diodes 445a-445c (labeled as “L” and collectively referred to as laser diodes 445). The laser diodes 445 are part of a triangulation positioning system configured to facilitate positioning of the cupula with respect to the target area. The plurality of laser diodes 445 may be disposed on the surface 448 equidistant from the central axis of the surface 448. In the illustrative example, the laser diodes 445 are positioned at the rim of the cupula. However, other locations on the surface 448 are possible. The laser diodes 445 may include three or more laser diodes, each of which may be a miniature low power visible spectrum laser diodes. The laser diodes 445 may be aligned such that light emitted from each of the diodes 445 converges at single focused spot at the focal plane of the cupula along the central axis. While not necessary, it may be preferential that the wavelength of beams emitted by the laser diodes 445 be in the visible spectrum to facilitate visualization by the operator. For example, while positioning the cupula relative to the target area, proper placement of the cupula may be determined when the target surface is positioned relative to the cupula such that the beams emitted from the laser diodes 445 converge at a single spot on the target surface. However, other wavelengths may be used in conjunction with corresponding detection techniques to detect proper alignment. In some embodiments, the laser power may be restricted to be 1 mW or less. As the laser diodes have their own distinct optical elements and the elements focus the beam on the cornea, when the cupula is properly positioned, none of the laser beams will be focused on the fovea of the eye together or singly thereby avoiding tissue damage even during an exposure of 24 hrs.

In an illustrative implementation, the irradiation head 410 may comprise 24 LEDs housed in a 75 mm focal length cupula. In a first example, each LED may be configured to emit green light (e.g., a peak wavelength at 525 nm) having a 15 degree half-angle. In this example, the irradiation head may produce an intensity of light measured as power density in watts per square meter up to 19 mW/cm². In a second example, each LED may be configured to emit UVA light (e.g., a peak wavelength at 375 nm). In this example, the irradiation head may produce an intensity of light measured as power density in watts per square meter up to 16 mW/cm². While the above embodiment is described with reference to an example configurations, the scope of this disclosure is not limited to these specific examples. For example, the number of light sources housed in the cupula and the power emitted thereby may be dependent on the size of the individual light sources and the size of the cupula and the desired incident power at the target tissue.

FIGS. 5A-5C illustrate an example irradiation head 510 in accordance with embodiment disclosed herein, but with the outer housing removed. The irradiation head 510 is substantively the same as irradiation head 310 and/or irradiation head 410. Therefore, the description above with respect to FIGS. 3A-4C apply equally to the embodiments shown in FIGS. 5A-5C, unless conflicting description is provided herein.

Similar to the above description, the irradiation head 510 includes a surface 548. As shown in the example of FIGS. 5A-5C, the surface 548 has a concave shape, however other shapes are possible as set forth above. Irradiation head 510 also comprises an image capture device 542 (e.g., similar to image capture devices 342 and/or 442) centrally mounted to the surface 548, through holes 520a-520c, and a cylindrical posts 510a-510c for receiving fasteners to mount the irradiation head 510 to the outer housing (e.g., outer housing 315 of FIG. 3A). The irradiation head 510 also comprises a plurality of coherent light sources 545a-545c (labeled as “L”) that are substantively the same as the coherent light sources 445 of FIGS. 4A-4C.

Additionally, the irradiation head 510 comprises a first plurality of light sources 544a-544n (labeled as “1” and collectively referred to as first light sources 544) and a second plurality of light sources 547a-547n (labeled as “2” and collectively referred to as second light sources 544) mounted to the surface 448. The first light sources 544 may be a first type of light source configured to emit light having a first peak wavelength to excite a first photosensitizer and the second light sources 547 may be a second type of light source configured to emit light having second peak wavelength to excite a second photosensitizer. The second type of light source may be different from the first type and the second peak wavelength may be different from the first peak wavelength. While two types of light sources are illustrated herein, it will be appreciated that the irradiation head 510 may comprise three or more different types of light sources, each emitting light of a different peak wavelength.

In the illustrative example of FIGS. 5A-5C, there are 18 of the first light sources 544 and 18 of the second light sources 547. However, the number of each type of light sources need not be the same. Additionally, the number of light sources may be fewer or greater than 18 as desired by the specific application.

Similar to the light sources described above, the first light sources 544 may be arranged in any desired array. The second light sources 547 may be arranged similarly. In some embodiments, the first and second types of light sources 544, 547 may alternate (e.g., a first light source is position between two second types along a given radius from the central axis and vice versa). In the illustrative example of FIG. 5B, the surface 548 is divided into four quadrants, each having a symmetrical arrangement of light sources 544 and 547. Additionally, as shown in FIG. 5B, subsets of first light sources may be grouped together forming a plurality of sets of first light sources disposed on the surface 548. Similarly, subsets of second light sources 547 may be grouped together between the subsets of the first light sources 544, with an alternating ring of first and second light sources 544, 547 closest to the image capture device 542. While FIG. 5B illustrates an example arrangement, the scope of the present disclosure encompasses any desired arrangement of first and second light sources.

As another example, the curvature of surface 548 may be varied as described above. In an illustrative arrangement, each subset of first and second light sources 544, 547 may be mounted at a respective region of surface 548, where each region of surface 548 has a different radius of curvature. Thus, a subset of light sources in a first quadrant of FIG. 5B and a corresponding subset of light sources in each of the other quadrants of FIG. 5B may be focused to a respective focal point. While, other subsets of light sources in each quadrant are focused to a different focal point based on the different radius of curvature of the surface 548.

While FIGS. 5A-5B illustrate the first plurality of light sources 544 and the second plurality of light sources 547 in a specific arrangement, any arrangement of the light sources may be possible. For example, the first light sources 544 and second light sources 547 may be arranged in alternating rows/columns of light sources or alternating rings of increasing radius from the central axis. As another example, each grouping (e.g., each row/column, ring, or array) may including one or more of each type of light source such that each grouping may include one or more first light sources 544 and one or more second light sources 547. Each type may be provided in an alternating manner or arranged in any desired arrangement.

Each group of light sources may be independently and separately controlled from each other so to excite different photosensitizers separately and independently during treatment. For example, the first light sources may be controlled to excite a first photosensitizer during a first phase of treatment and the second light source controlled to excite a second photosensitizer in a second phase that is either before or after the first phase.

FIGS. 6A-6D illustrates an example graphical user interface (GUI) 600 for controlling the PDT system 200 in accordance with embodiments disclosed herein. The graphical user interface 600 may be generated by a controller of a PDT system as described herein, for example, controller 220 of system 200 of FIG. 2. The graphical user interface may be generated by the controller and displayed to the operator via a display, such as user interface 225 implemented as a touch screen display. From the GUI 600, photosensitizer soaking phase and irradiation phase parameters can be set by the treating physician and/or operator.

The GUI 600 comprises a plurality of regions, including but not limited to a, phase region 607, a dynamic region 605, and a imaging region 620. Phase region 607 displays controls for a selected phase of the treatment procedure. Region 607 includes a plurality of tabs, each tab corresponding to a different phase. For example, a patient identification phase at tab 610 as shown in FIG. 6A, a pre-treatment phase (also referred to as a photosensitizer soaking phase) at tab 630 as shown in FIG. 6B, PDT phase (also referred to as a photosensitizing phase) at tab 640 as shown in FIG. 6C, and a post-treatment phase at tab 650 as shown in FIG. 6D. Region 607 displays input icons for entering parameters and criteria for controlling the system 200 at each phase, with different icons displayed based on the selected phase. The dynamic region 605 is changed automatically based on which phase is selected in the phase region 607. Similar to the phase region, region 605 displays input icons for controlling the system 200 based on the selected phase. Thus, region 605 may function as an overflow for additional control icons. Region 620 generates a display of the image or video stream captured for the target tissue (e.g., the ocular surface of eye 622 in this example), for example, by the image capture device 342 of the PDT device 300. From the region 620, the operator can monitor alignment and treatment throughout the procedure. Each region may be provided in the same window (or display pane) or may be displayed in separate display panes arranged and/or stacked windows.

The GUI 600 includes a plurality of icons on which the operator may interact with to input parameters and controls for controlling the PDT system. Where the GUI 600 is displayed on a touch screen, the icons may be interacted with via the touch screen (e.g., touching gestures, swiping gestures, etc.), to input parameters. In other examples, the operator may control a pointer (e.g., via a mouse or other user input device) to interact with icons and input parameters via a keyboard. In another example, the operator may control inputs using voice commands and voice recognition technology.

In the illustrative example, region 620 is displaying an image (or video stream) of an eye 622 that is positioned for treatment (e.g., an eye of user 250). Also illustrated in region 620 is a sponge 626 (e.g., a sterile sponge) that can be or is soaked with a selected photosensitizer for treatment. In some embodiments, the sponge may be soaked with multiple photosensitizers. In this case, it may be desirable to ensure the multiple photosensitizers do not interact chemically to cancel or reduce effectiveness of the photosensitizers. Region 620 also comprises an indicator 624 of the treatment region of interest (ROI). Indicator 624 indicates the ROI in which light is to be emitted from the PDT device 300. The tissue located within the ROI will be exposed to constant energy levels of radiation (e.g., light) emitted by the PDT device 300. For example, a constant 6 mW/cm² in the illustrative example described in connection to FIG. 3B; a constant 19 mW/cm²m as described in connection to FIGS. 4A-4C; and/or a constant 16 mW/cm² as described in connection to FIGS. 4A-4C. These power levels are examples only, and other power levels are possible. In the illustrative example, the controller generates a circle as an indicator 624 (shown as a dotted line in FIGS. 6A-6D). However, any visual indication would be applicable such that the indicator 624 corresponds to the ROI. The region 620 may also display energy metrics for the irradiation during the corresponding phase of treatment.

In some examples, the operator may assess the size of the target tissue (e.g., corneal infiltrate) and configures the device for an optimal treatment. For example, the operator may input a desired ROI in the region 620 based on the assessment and the controller may generate the indicator 624 based on the input. In another example, the software may analyze and assess the dimension and location of target tissue, and then generate a recommendation for a preferred photosensitizer, treatment duration and intensity. The recommendation may be verified by the operator and updated as needed based on manual review.

FIG. 6A illustrates an example GUI 600 in the patient ID phase at tab 610. From this tab, an operator can enter patient identifying information, such as a patient code number, age, gender, onset time, visit number, eye information, etc. Also, from this screen, the operator can enter microbes present in the target tissue if known (e.g., fungal: yeast; fungal: mold; bacteria: gram+; bacteria: gram-; amoeba; mycobacteria; nocardia; microsporidia; etc.) or enter unknown. The operator may input suspected or identified microorganism and the controller can be programmed to recommend an ideal wavelength and irradiation head (based on light sources therein) to use, and establish optimal treatment durations. Additionally, from this screen, the operator may enter known risk factors (e.g., O.S.D.; trauma; steroid use; contact lens; etc.) or none.

In some implementations, the operator may select an irradiation head based on the photosensitizer to be applied (e.g., a head that emits light to excite the applied photosensitizer). In implementation, the controller may detect the photosensitizer applied (either through automated detection or manual input) and confirms that the corresponding irradiation head is connected. For example, the controller may detect the irradiation head is coupled to the support via known techniques of detecting coupling of two bodies. Based on the detection, the controller may receive identifying information of the irradiation head (either from operator input or data exchange with the irradiation head) and then confirm that the light sources housed therein will emit the proper peak wavelength. Confirmation may be based, for example, on a look up table listing identification information (e.g., part numbers or the like) of the irradiation head and specification identifying the emitted light for each irradiation head in the look up table. As another example, the irradiation head may transmit information indicating the wavelength to the controller.

In yet another example, the irradiation head and photosensitizer have a “lock and key” mechanism to ensure that only the correct head can be used with the appropriate photosensitizer. For example, one of wires 245, 240 may include a multi female receptacles socket coupled to the controller 220 that can accept different irradiation heads. Each irradiation head may have a multi prong male connector that mates with the multi female receptacle socket for establishing electrical connections with the controller 220, but each irradiation head may have a pin that indicates to the controller 220 what wavelength(s) the irradiation head is designed to produce. The controller 220 may then determine what wavelength(s) a respective irradiation head is able to produce, identify the proper voltage and software for the particular irradiation head, and generate and apply the voltage and display the software designed for that particular irradiation head (e.g., lock and key). Each irradiation head comprises a plurality of light sources, but not all will have the same number, types, etc. of light sources. Thus, the voltage and current required to drive each irradiation head may vary and the above example ensures the controller 220 recognizes which irradiation head it is connected.

FIG. 6B illustrates an example GUI 600 in the pre-treatment phase at tab 630. At this phase, the sponge 626 may be soaked with a selected photosensitizer. From this screen, the operator can set soak duration, adjust the position of the PDT device 300 (e.g., via manual movement or through remote operation via region 605), and set photosensitizer dose reminder time intervals (e.g., 2 or 3 minutes). Based on the selected reminder interval, an audible or visual reminder will be issues when the next dose of photosensitizer needs to be applied to the sponge. In some implementations, the operator may manually apply drops of the photosensitizer on the target surface or on the sponge 626 positioned on the defect. In another implementation, the photosensitizer may be automatically delivered by the device via a nozzle included in the irradiation head, with variable and/or continuous dosing. From display region 620, the soaking procedure and target tissue may be monitored. Additionally, the operator can capture images and record video from icons in tab 630 and control the zoom of image capture device 342, which is translated to display region 620.

In some embodiments, the irradiation head may be calibrated during manufacturing. To confirm calibration, the system 200 (or PDT device 300 when removed from system 200) may be docked at a remote station that measures the optical power output before each treatment. However, in another iteration, the irradiation head may comprise an integrated power meter to auto calibrate and track operating specification range. Calibration tracking and confirmation may be displayed on the GUI 600 during the pre-treatment phase in tab 630 (not shown).

FIG. 6C illustrates an example GUI 600 in the PDT phase at tab 640. From this screen, the operator can control and monitor the irradiation procedure. Icons in FIG. 6C, in region 605, facilitate control of the irradiation energy via the toggle icons on the left side of region 605. When the operator selects an energy level above a predetermined threshold (e.g., based on medical standard, such as 10 mw/cm² in this example) the background of the energy level may change color (for example, from green to orange). This background is shown as diagonal lines in FIG. 6C indicating that the level as been set above the threshold. Other display attributes may be utilized.

In some implementations, the energy level delivered by the system 200 may be fixed and the operator may adjust treatment duration from the GUI 600 of FIG. 6C. In another example, both the power level and duration may be configurable by the operator. In still another example, the operator may adjust the power level, and the ideal treatment duration may be calculated by the controller and tracked.

When the power level is adjusted by the operator, the output may be maintained in a stable state throughout the PDT phase. In another example, the user can select for the power to oscillate in multiple patterns (i.e. sine, square, pulsed, triangle) via tab 640. By interacting with the “Oscillate Off” icon, an oscillate control interface may be generated (e.g., in region 605 or another window) for controlling the oscillation pattern and frequency. Photosensitizer energy delivered in variable patterns based on the selected oscillation, may improve patient comfort and improve conditions for the production of oxygen radicals.

In some embodiments, the operator may input optimal power level for the treatment via tab 640. In another example, the system 200 starts the treatment phase at a low power level, and slowly ramps the power until the patient reports a slight discomfort level. The system 200 may then reduce the power to a level where the patient does not experience discomfort (e.g., a no discomfort level) and calculates the treatment duration at this ideal comfort level.

A session timer is provided that displays the duration of the treatment and an elapsed time timer is provided to monitor time of a given irradiation. From the positing system icon, the operator can access the positioning system to ensure proper alignment throughout treatment.

In some implementations, the controller may estimate the level of oxygen radical to be produced based on output power levels and treatment duration parameters. The light intensity and treatment duration may be automatically adjusted to create optimal levels of oxygen radicals based on the selected photosensitizer and estimate oxygen radical production. In some implementations, an optimizing gases (e.g., oxygen, nitrogen, air) may be circulated over the target area to improve efficacy of the oxygen radical production. The controller 200 may adjust gas flow to maintain optimal production of oxygen radicals. For example, a nozzle attached to a reservoir of optimizing gas may be coupled to the controller 200. The controller 200 may control a flow gauge to control the gas flow based on maintaining optimal production of oxygen radicals.

During treatment, light may be emitted axially to the target tissue, for example, the enteral axis 312 is positioned substantially perpendicular to the target area. As another example, where the target is an eye, the light may be applied axially to the cornea. In some cases, other directions of incidence may be utilized, for example, light may be applied at different angles relative to the target area. In some embodiments, the support 210 may be manually moved to different angels, while in another example, the support 210 may be a robotic arm that moves the PDT device 300. Different angels of incidence may provide greater amounts of irradiation to the infected area; reduce the irradiation to neighboring healthy tissues; and reduce the patient's discomfort related to light exposure.

FIG. 6D illustrates an example GUI 600 in the post-treatment phase at tab 650. Having completed treatment, the operator may control the device to capture images of the target tissue to confirm treatment completion. A reminder may be generated as to a number of images to capture.

FIG. 7 is a functional block diagram of one embodiment of an exemplary controller in the form of a computer system 700 which can be used as part of the various embodiments described here. The computer system 700 may be implemented as the controller 220 of FIG. 2A in various embodiments. The computer system 700 includes a controller 710 having one or more processors 715, a memory 720, storage 730, a user interface 750 (See FIGS. 6A-6D for examples of graphical user interfaces), an input/output (I/O) interface 760, and a network interface 770. These components are interconnected by a common bus 780. Alternatively, different connection configurations can be used, such as a star pattern with the controller at the center.

The controller 710 is a programmable processor and controls the operation of the computer system 700 and its components. The controller 710 loads instructions from the memory 720 or an embedded controller memory (not shown) and executes these instructions to control the system as described above. These instructions may cause the controller 710 to perform the functions described throughout this disclosure, for example, in connection to FIGS. 1-6D.

Memory 720 stores data temporarily for use by the components of the computer system 700. In one embodiment, memory 720 is implemented as RAM. In one embodiment, memory 720 also includes long-term or permanent memory, such as flash memory and/or ROM.

Storage 730 stores data temporarily or long term for use by the components of the computer system 700. In one embodiment, storage 730 is a hard disk drive. Storage 730 stores information for use by the one or more processors 715. Storage 730 also stores data generated by one or more processors 715.

The user interface 750 includes components for accepting user input from a user of the computer system 700 and presenting information to the user. In one embodiment, the user interface 750 includes a keyboard, a mouse, audio speakers, and a display. The controller 710 uses input from the user to adjust the operation of the computer system 700. The user interface 720 may be an example of user interface 225 of FIG. 2A.

The I/O interface 760 includes one or more I/O ports to connect to corresponding I/O devices, such as the laser source(s) and the robotic arm referenced above. In various embodiments, the ports of the I/O interface 760 include ports such as: USB ports, PCMCIA ports, serial ports, and/or parallel ports. In another embodiment, the I/O interface 760 includes a wireless interface for communication with external devices wirelessly.

A network interface 770 can be included as a wired and/or wireless network connection, such as an RJ-45 or “Wi-Fi” interface (802.11) supporting, for example, an Ethernet connection.

The computer system 700 includes additional hardware and software typical of computer systems (e.g., power, cooling, operating system), though these components are not specifically shown in FIG. 7 for simplicity. In other embodiments, different configurations of the computer system can be used (e.g., different bus or storage configurations or a multi-processor configuration).

Those of skill in the art will appreciate that the various illustrative logical blocks, modules, circuits, and steps described in connection with the above described figures and the embodiments disclosed herein can often be implemented as electronic hardware, computer software, or combinations of both. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA, or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A device for photodynamic therapy treatment of a target biological tissue having an infection, the device comprising:
an irradiation head comprising a first end and a surface at a second end, the surface having a radius of curvature; and
a plurality of light sources disposed on the curved surface, the plurality of light sources configured to emit light having at least one wavelength corresponding approximately to an excitation peak of at least one photosensitizer, wherein light emitted by the plurality of light sources is focused to a focal point based on the radius of curvature of the surface.

2. The device of claim 1, wherein the surface is a concave surface forming a cupula structure.

3. (canceled)

4. The device of claim 1, wherein the at least one photosensitizer is selected from the group consisting of Rose Bengal, Erythrosin B, riboflavin, dihematoporphyrinether, verteporfin, and Methylene Blue.

5. The device of claim 1, wherein light emitted from each of the plurality of light sources is focused on to the target biological tissue positioned at the focal point of the radius of curvature, wherein the at least one photosensitizer is selected to treat the infection of the target biological tissue.

6. The device of claim 1, wherein the radius of curvature is varied.

7. The device of claim 2, further comprising a plurality of coherent light sources disposed on the curved surface equidistant from each other and each arranged to emit a beam toward the focal point.

8. The device of claim 7, wherein the plurality of coherent light sources are included as part of a positioning system, the positioning system configured to track a position of the irradiation head relative to the target biological tissue based on the convergence of the beams emitted from the coherent light sources on the target biological tissue.

9. The device of claim 8, wherein the coherent light sources emit light having a wavelength in the visible spectrum.

10. (canceled)

11. The device of claim 1, wherein the at least one wavelength is selected from the group consisting of approximately 375 nm, approximately 400 nm, approximately 670 nm, and approximately 395 nm.

12. The device of claim 1, wherein the plurality of light sources comprises a first plurality of light sources and a second plurality of light sources, the first plurality of light sources configured to emit light having a first wavelength corresponding approximately to an excitation peak of a first photosensitizer and the second plurality of light sources configured to emit light having a second wavelength corresponding to approximately an excitation peak of a second photosensitizer, second photosensitizer being different from the first photosensitizer.

13. The device of claim 12, wherein the first and second wavelengths are selected from the group consisting of approximately 375 nm, approximately 400 nm, approximately 670 nm, and approximately 395 nm.

14. (canceled)

15. (canceled)

16. (canceled)

17. A photodynamic therapy system comprising:
the device according to claim 1; and
a controller communicatively coupled to the plurality of light sources and configured to control the light emitted by the plurality of light sources.

18. The system of claim 17, further comprising a support arm, wherein the irradiation head is coupled to the support arm.

19. (canceled)

20. The system of claim 18, wherein the support arm is a robotic arm, wherein the robotic arm is communicatively coupled to the controller and the controller is configured to control the robotic arm and position the irradiation head with respect to a target tissue for treatment.

21. The system of claim 17, wherein the controller is configured to generate a graphical user interface for display on a user interface, the graphical user interface comprising a plurality of screens each corresponding to a phase of treatment, each screen comprising a user interface for receipt of parameters to control the photodynamic therapy system.

22. A method of treating a condition of a target biological tissue, the method comprising:
deploying a photodynamic therapy system according to claim 17;
positioning the device relative to the target biological tissue such that the target biological tissue is positioned at the focal point of the radius of curvature;
applying the at least one photosensitizer to the target biological tissue, the at least one photosensitizer having the excitation peak; and
controlling the plurality of light sources, using the controller, to emit light having the at least one wavelength toward the photosensitizer, thereby exciting the photosensitizer and producing oxygen radicals.

23. A method of treating a condition of an eye, the method comprising:
applying at least one photosensitizer to the cornea of an eye, the photosensitizer having an excitation peak;
positioning a corneal surface of the eye at a focal point of a cupula; and
applying at least one wavelength of light to the cornea of the eye for a predetermined period of time using a plurality of light sources emitting light having the at least one wavelength that corresponds approximately to the excitation peak of the at least one photosensitizer, the plurality of light sources housed in the cupula structure, the cupula structure having a radius of curvature that focuses the light emitted from each of the plurality of light sources at the focal point and on to the corneal surface of the eye positioned at the focal point of the cupula.

24. The method of claim 23, wherein the at least one photosensitizer is selected from the group consisting of Rose Bengal, Erythrosin B, riboflavin, dihematoporphyrinether, and verteporfin.

25. A device for photodynamic therapy, the device comprising:
a cupula structured member having a radius of curvature; and
a plurality of light sources positioned within a concave surface of the cupula structured member, each of the plurality of light sources configured to emit light of a wavelength that is common amongst the plurality of light sources, the wavelength corresponding to an excitation peak of a photosensitizer;
wherein the radius of curvature of the cupula focuses light emitted by the plurality of light sources to a focal point.

26. The device of claim 26, further comprising another plurality of light sources configured to emit light at another wavelength that is the same amongst the other plurality of light sources, the other wavelength corresponding to approximately an excitation peak of another photosensitizer.
